(19) United States
(12) Patent Application Publication
(10) Pub. No.: US 2024/0400963 A1
(43) Pub. Date: Dec. 5, 2024

(54) HYDROSTATIC DEVICE FOR CELL DIFFERENTIATION

(71) Applicant: THE ROYAL INSTITUTION FOR THE ADVANCEMENT OF LEARNING / MCGILL UNIVERSITY, Montréal (CA)

(72) Inventors: Christopher MORAES, Montreal (CA); Chen LI, Montreal (CA)

(21) Appl. No.: 18/733,250

(22) Filed: Jun. 4, 2024

Related U.S. Application Data
(60) Provisional application No. 63/471,086, filed on Jun. 5, 2023.

(51) Int. Cl. C12M 1/12; C12M 1/00; C12M 1/06

(52) U.S. Cl. CPC C12M 25/02; C12M 23/34; C12M 23/38; C12M 27/06; C12M 29/14

(57) ABSTRACT

A device for cell differentiation, which may be used for culturing cells for example, includes an enclosure defining a liquid-receiving volume for receiving a liquid media, and an insert received in the enclosure. The insert includes a peripheral wall circumscribing an apical volume and having an opening in a bottom end of the insert. A membrane extends across the opening and defines pores sized to receive cells, with an apical side of the membrane adapted to receive cells thereon. The membrane and the peripheral wall separate the liquid-receiving volume into an apical volume within the insert and a basal volume below the membrane within the enclosure. A source of a pressurized fluid is in fluid flow communication with the basal volume to define a basal pressure in the basal volume that is greater than an apical pressure in the apical volume acting against the apical side of the membrane.

### CROSS-REFERENCE TO RELATED APPLICATIONS

This application claims priority benefit from U.S. provisional application No. 63/471,086 filed on Jun. 5, 2023, the entire contents of which are incorporated by reference herein.

### TECHNICAL FIELD

The present disclosure relates generally to biological cell culturing and, more particularly, to systems and methods used to culture and differentiate biological cells.

### BACKGROUND

Biological cells can be differentiated to have specialized functions to build realistic in vitro cell culture models. The methods to achieve this are typically based on chemical stimulation with multiple differentiation factors and steps, but can also incorporate biophysical cues such as subjecting cells to specific forces, or combinations of these signals by engineering specific microenvironments.

The use of an Air-liquid interface (ALI) is a well-established cell culturing method to differentiate epithelial cells (cells lining the inner and outer surfaces of the human body) towards functional phenotypes and allows scientists to generate realistic tissue/organ models for research purposes. For example, ALI is used to grow tracheobronchial epithelium which closely mimics human airway tissues for drug screening or studying lung diseases. Despite common use of ALI for many applications, the precise causes of differentiation underlying this approach remains unknown. This lack of knowledge creates additional challenges: ALI differentiation typically requires long culture times (multiple weeks), and the use of porous inserts which are specialized and challenging to handle. These difficulties make ALI culture prone to handling errors which limit scaling up for high-throughput applications. Improvements are therefore sought.

### SUMMARY

There is accordingly provided a device for cell differentiation, comprising: an enclosure having a top and a bottom, the enclosure defining a liquid-receiving volume for receiving a liquid media; an insert received in the enclosure, the insert having: a peripheral wall circumscribing an apical volume and extending from a top end proximate the top of the enclosure to a bottom end, an opening defined at the bottom end of the insert, and a membrane extending across the opening and defining pores sized to receive cells, the membrane having a basal side facing the bottom of the enclosure and an apical side opposite the basal side, the apical side adapted to receive cells thereon, the membrane and the peripheral wall separating the liquid-receiving volume into an apical volume within the insert and a basal volume below the membrane within the enclosure; and a source of a pressurized fluid in fluid flow communication with the basal volume within the enclose, the pressurized fluid being at a pressure selected such that a basal pressure in the basal volume acting against the basal side of the membrane is greater than an apical pressure in the apical volume acting against the apical side of the membrane.

The device(s) defined above and described herein may also include any one or more of the following features, in whole or in part, and in any combination.

In certain embodiments, the source of the pressurized fluid is a reservoir of the liquid media, the reservoir in fluid flow communication with the basal volume, a first elevation of the liquid media in the reservoir being greater than a second elevation of the liquid media in the apical volume.

In certain embodiments, the reservoir is engaged to an actuator operable to vary the elevation of the reservoir.

In certain embodiments, the actuator includes a motor drivingly engaged to a threaded shank and a member threadingly engaged to the threaded shank, the reservoir mounted to the member, rotation of the threaded shank with the motor inducing a translation of the member and the reservoir about a rotation axis of the threaded shank.

In certain embodiments, the actuator includes a motor drivingly engaged to a rotating member for rotation about a rotation axis, the reservoir mounted to the rotating member, the rotating member having a pin secured thereto and offset from the rotation axis of the rotating member, the pin slidably received within a slot defined by the member, rotation of the rotating member with the motor induces a translation of the member and the reservoir about an axis normal to the rotation axis of the rotating member.

In certain embodiments, the source of the pressurized fluid is a compressor fluidly connected to the basal volume and configured for increasing an air pressure in the basal volume.

In certain embodiments, the enclosure defines an outlet in fluid flow communication with the basal volume.

In certain embodiments, a valve is in fluid flow communication with the outlet, the valve configured for selectively fluidly connected the basal volume to an environment outside the basal volume.

In certain embodiments, the insert includes a plurality of inserts disposed within the basal volume of the enclosure, each of the plurality of inserts defining a respective apical volume.

In certain embodiments, two or more of the plurality of inserts have different basal volumes, such as to induce different pressures pressure on a basolateral side of the membrane.

In certain embodiments, a top plate is secured to the top of the enclosure, the top plate defines apertures, the plurality of inserts received through the apertures.

In accordance with another aspect, there is also provided a method for culturing cells, comprising: exposing an apical side of a membrane having cells adhered thereto to a first fluid pressure; and exposing a basal side of the membrane opposite the apical side to a second fluid pressure different than the first fluid pressure.

The method defined above and described herein may also include any one or more of the following features, in whole or in part, and in any combination.

In certain embodiments, the second fluid pressure is greater than the first fluid pressure.

In certain embodiments, the method includes, prior to the exposing the basal side of the membrane to the second fluid pressure, exposing both the apical side and the basal side to media at substantially equal pressure, determining when a cell layer on the membrane becomes confluent, and then exposing the basal side of the membrane to the second fluid pressure.

In certain embodiments, the membrane extends across an opening defined by an insert received within a fluid-receiving volume of an enclosure, the membrane dividing the fluid-receiving volume in an apical volume and a basal volume below the apical volume, the exposing of the basal side of the membrane to the second fluid pressure includes fluidly connecting the basal volume to a source of a pressurized fluid.

In certain embodiments, fluidly connecting of the basal volume to the source of the pressurized fluid includes fluidly connecting the basal volume to a reservoir of a liquid media, a first elevation of the liquid media in the reservoir being greater than a second elevation of the liquid media in the apical volume.

In certain embodiments, fluidly connecting of the basal volume to the source of the pressurized fluid includes fluidly connecting the basal volume to a compressor.

In certain embodiments, the method includes cyclically varying the second fluid pressure.

In certain embodiments, cyclically varying the second fluid pressure includes cyclically varying an elevation of the reservoir.

In certain embodiments, exposing of the apical side of the membrane to the first fluid pressure and the exposing of the basal side of the membrane to the second fluid pressure includes exposing a plurality of apical sides of a plurality of membranes to the first fluid pressure and exposing a plurality of basal sides of the plurality of membranes to the second fluid pressure.

In one specific implementation, a custom removable acrylic well plate adapter is provided and produced, for example, by laser cutting to position an array of fluid inserts (e.g., Transwell™ inserts) in a well plate and create a common sealed bottom compartment ready for pressurization. A tall thin tube planted vertically in the well plate bottom compartment is used to induce hydrostatic pressure with minimal use of liquid. The device may be used to culture a primary lung cell line commonly cultured in ALI condition, human bronchial epithelial (HBE) cells, and a miniature in vitro gut organ each in 3 different conditions: control (submerged in media and no pressure difference), ALI, and pressured (using the built platform) for up to 3 weeks (differentiation time required for traditional ALI culture) with samples analyzed at the end of each week to create a differentiation timeline.

### BRIEF DESCRIPTION OF THE DRAWINGS

Reference is now made to the accompanying figures in which:

FIG. 1 is a schematic view of a device for cell differentiation in accordance with one embodiment;

FIG. 2 is a schematic view of a device for cell differentiation in accordance with another embodiment;

FIG. 3 is a schematic view of a device for cell differentiation in accordance with another embodiment;

FIG. 4 illustrates a reservoir for containing a liquid media used for cell differentiation and used for generating hydrostatic pressure;

FIG. 5 is another view of the reservoir containing two different liquids;

FIG. 6 illustrates two reservoirs used for supplementing the liquid media to the device of FIGS. 1-3 and for generating the hydrostatic pressure;

FIG. 7 is a schematic view of a device for cell differentiation in accordance with yet another embodiment;

FIG. 8 illustrates a system for varying the hydrostatic pressure of the reservoir of FIG. 4;

FIG. 9 illustrates another system for varying the hydrostatic pressure of the reservoir of FIG. 4;

FIG. 10 is a flowchart illustrating steps of a method of creating cell differentiation;

FIGS. 11A and 11B illustrate cell growth on lung (FIG. 11A) and gut (FIG. 11B) tissue when no pressure differential is applied on the membrane containing the cells;

FIGS. 12A and 12B illustrate cell growth on lung (FIG. 12A) and gut (FIG. 12B) tissue when a pressure differential is applied on the membrane;

FIG. 13A is a three dimensional view of an adaptor in accordance with one embodiment for sealing a reservoir containing a liquid media;

FIG. 13B is a cross-sectional view of the adaptor of FIG. 13A;

FIG. 13C is a cross-sectional view illustrating the adaptor of FIG. 13A received within a reservoir and receiving an insert used for cell differentiation;

FIG. 14A is a three dimensional view of an adaptor in accordance with another embodiment for sealing a reservoir containing a liquid media;

FIG. 14B is a cross-sectional view of the adaptor of FIG. 14A;

FIG. 14C is a cross-sectional view illustrating the adaptor of FIG. 14A received within a reservoir and receiving an insert used for cell differentiation;

FIG. 15A is a top three dimensional view of a lid extender in accordance with one embodiment;

FIG. 15B is a side three dimensional view of the lid extender of FIG. 15A;

FIG. 15C is a cross-sectional view of the lid extender of FIG. 15A;

FIG. 16A is a three dimensional partially transparent view of a device in accordance with yet another embodiment; and

FIG. 16B is a three dimensional exploded view of the device of FIG. 16A.

### DETAILED DESCRIPTION

Air-liquid interface (ALI) consists of culturing cells on a porous membrane, while allowing them to access nutrient-containing culture media, which may be simply referred to herein as the liquid of the liquid media, through the porous membrane and exposing the top surface of cells to air. Inventors of the present disclosure attempted to recreate these ALI conditions by growing epithelial cells on a media-soaked hydrogel exposed to air from the top surface. Unexpectedly, the ALI differentiation did not occur. The caused hypothesized by the inventors of the present disclosure is that the innate hydrostatic biophysical pressure difference created by ALI across the epithelial barrier might play a critical role in epithelial differentiation.

Understanding the mechanism of ALI epithelial differentiation may allow the development of a more flexible and robust differentiation method that may permit scaling up the fabrication process of in vitro cell culture models.

Inventors of the present disclosure discovered that the common ALI culture model is driven by an inherent pressure differential across the epithelial membrane barrier. This finding might thus be the key in determining the differentiation mechanism since it could be used as a “filter” condition during genetic analysis. On comparison with the full ALI condition, the specific and critical genes involved in differentiation will likely be more apparent compared to the existing genetic analysis of ALI differentiation.

The present disclosure describes a device used to subject the cell layer to a pressure differential for cell differentiation, while maintaining cells at standard submerged culture.

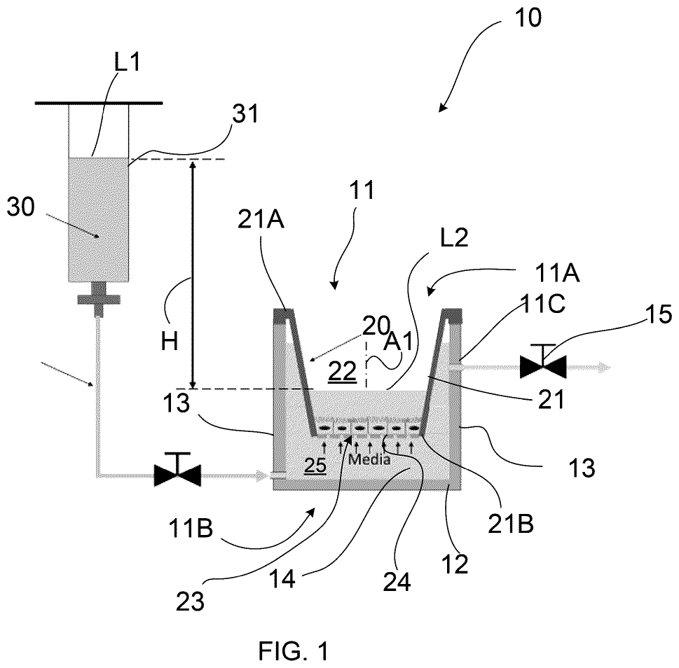

Referring to FIG. 1, a device used for cell differentiation is shown at 10. The device 10 includes an enclosure 11 for receiving a liquid media used for feeding the cells. The enclosure 11 includes a top 11A and a bottom 11B. The expressions “top” and “bottom” are relative to a vertical direction perpendicular to a ground. The enclosure 11 includes a bottom wall 12 at the bottom 11B and the top 11A is open. The enclosure 11 includes one or more interconnected side walls 13 secured to the bottom wall 12 and protruding upwardly therefrom towards the top 11A. The side walls 13 and the bottom wall 12 define a liquid-receiving volume 14 for receiving the liquid media used for cell differentiation. The enclosure 11 may have any suitable shape, such as cylindrical, square, rectangular, and so on.

The device 10 includes an insert 20. It may include more than one insert 20 and, for the sake of clarity, the description below uses the singular form and may apply to all inserts. The insert 20 is received within the liquid-receiving volume 14 of the enclosure 11. In other words, the insert 20 is located between the top 11A and the bottom 11B of the enclosure 11 and is meant to be at least partially submerged by the liquid media contained in the liquid-receiving volume 14. The insert 20 has a peripheral wall 21 that extends around a central axis A1 of the insert 20. The peripheral wall 21 has a frustoconical shape in this embodiment, but other shapes, such as cylindrical, rectangular, and so on are contemplated. The peripheral wall 21 defines a cross-section taken on a plane normal to the central axis A1. An area of this cross-section may decrease towards the bottom wall 12 of the enclosure 11.

The peripheral wall 21 extends from a top end 21A to a bottom end 21B. The top end 21A is proximate the top 11A of the enclosure 11 whereas the bottom end 21B is proximate the bottom 11B of the enclosure 11. In the embodiment shown, the top end 21A of the peripheral wall 21 is secured to a top edge of the side walls 13 of the enclosure 11 whereas the bottom end 21B of the peripheral wall 21 is spaced apart from the bottom 11B of the enclosure 11. A sealing engagement may be provided between the insert 20 and the enclosure 11. The sealing engagement is defined between the top end 21A of the peripheral wall 21 of the insert 20 and the top 11A of the enclosure 11. The insert 20 may hang from the top 11A of the enclosure 11. The insert 20 circumscribes an apical volume 22 that is located axially between the top end 21A and the bottom end 21B of the peripheral wall 21. An opening 23 is located at the bottom end 21B of the peripheral wall 21.

The insert 20 further includes a membrane 24 extending across the opening 23 and defining pores sized to receive cells therethrough. The membrane 24 fluidly separates the apical volume 22 from a remainder of the liquid-receiving volume 14 of the enclosure 11. Once the cells have filled the pores of the membrane 24, the membrane 24 becomes effectively watertight. The membrane 24 has a basal side facing the bottom 11B of the enclosure 11 and an apical side opposite the basal side and facing the apical volume 22. The membrane 24 and the peripheral wall 21 separates the liquid-receiving volume of the enclosure 11 in a basal volume 25 below the membrane 24 and the apical volume 22 above the membrane 24.

The device 10 is configured for creating a pressure differential between opposite sides, namely the apical side and the basal side, of the membrane 24. The device 10 thus uses a source of a pressurized fluid 30 in fluid flow communication with the basal volume 25 and at a pressure selected such that a pressure in the basal volume 25 against the basal side of the membrane 24 is greater than a pressure in the apical volume 22 against the apical side of the membrane 24.

As shown in FIG. 1, the source of the pressurized fluid may be a reservoir 31 of the liquid media. The reservoir 31 is in fluid flow communication with the basal volume 25 and is located at an elevation greater than an elevation of the top 11A of the enclosure 11. Put differently, an elevation of a first level L1 of the liquid media in the reservoir 31 is greater than an elevation of a second level L2 of the liquid media above the membrane 24 in the apical volume 22. An elevation difference H between the first level L1 and the second level L2 is correlated to the pressure difference exerted on the opposite sides of the membrane 24.

In the embodiment shown, the enclosure 11 defines an outlet 11C in fluid flow communication with the basal volume 25. A valve 15 is in fluid flow communication with the outlet 11C and has open and closed configurations to either permit the liquid to flow out of the basal volume 25 through the valve 15 or to prevent the liquid from flowing out of the basal volume 25. The valve 15 may be open to permit a replacement and/or addition of the liquid in the basal volume 25. In other words, after some time it may be required to replace or supplement the liquid in the basal volume 25. To do so, the valve 15 may be opened thereby permitting fluid flow communication from the reservoir 31 of the liquid to the basal volume 25 and from the basal volume 25 through the valve 15. New liquid may then be added to the reservoir 31.

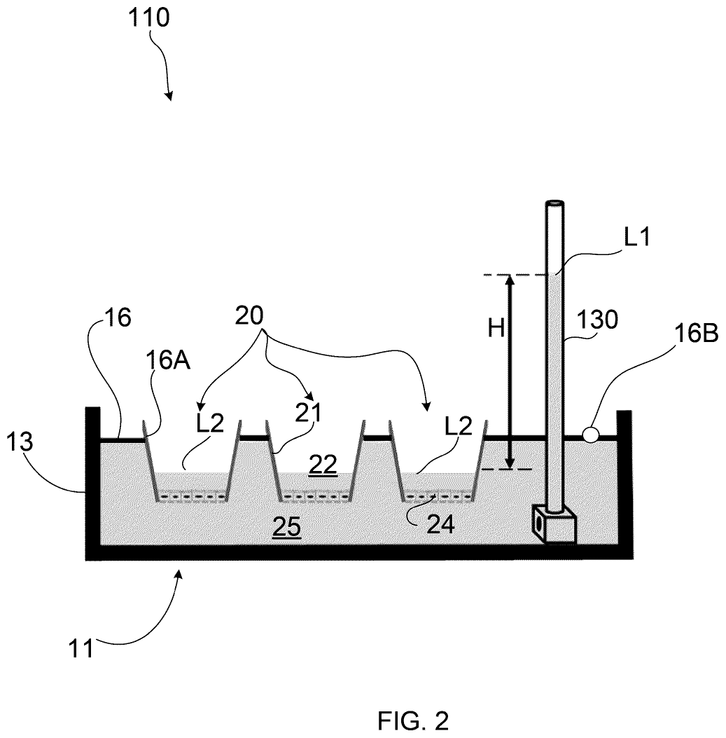

Referring now to FIG. 2, another embodiment of a device for cell differentiation is shown at 110. For the sake of conciseness, only features differing from the device 10 of FIG. 1 are described below.

In the embodiment illustrated, the device 110 includes a plurality of inserts 20. Although three inserts 20 are illustrated, less or more than three inserts 20 may be used. A top plate 16 may be secured to the one or more side walls 13 and defines a plurality of apertures 16A each sized to receive a respective one of the inserts 20 there through. A sealing engagement is defined between the top plate 16 and the side walls 13. A sealing engagement may be defined between the inserts 20 and the top plate 16.

In this embodiment, the source of the pressurized fluid is a tube 130 having a first end in fluid communication with the basal volume 25 and a second end open to atmospheric pressure. The tube 130 extends at an elevation greater than an elevation of the top 11A of the enclosure 11. This allows the tube 130 to be filled with the liquid at a first level L1 greater than an elevation of a second level L2 of the liquid above the membrane 24 in the apical volume 22. An elevation difference H between the first level L1 and the second level L2 is correlated to the pressure difference exerted on the opposite sides of the membrane 24.

A vent 16B may be defined through the top plate 16 and used to allow air to exit the basal volume 25 during the filling of the basal volume 25 with the liquid. The vent 16B may be sealable to prevent the liquid from leaking out of the basal volume 25. The vent 16B may alternatively be defined through the peripheral wall 21 of the insert 20 and above the second level L2 of liquid in the apical volume 22.

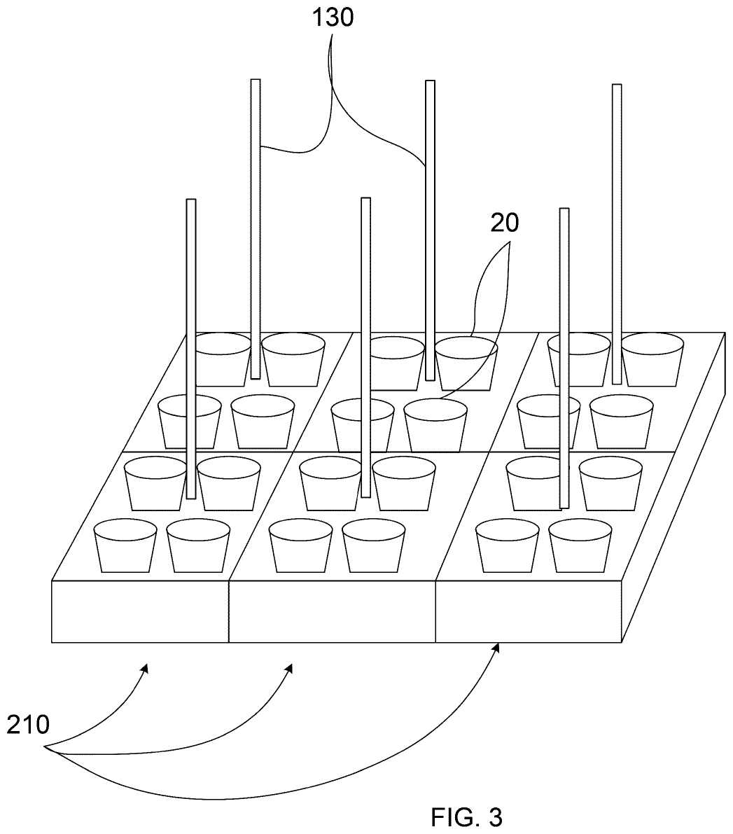

Referring now to FIG. 3, a plurality of devices for cell differentiation may be assembled in a matrix. In this embodiment, each devices 210 includes an enclosure 11 and four inserts 20, although more or less than four inserts 20 may be used. Each devices 210 has a respective source of pressurized fluid, which corresponds to a tube 130 as described in FIG. 2. In this configuration, each devices 210 may subject its respective membranes to a respective pressure since the level of liquid in the tubes 130 may be varied from one device 210 to the other. Each device 210 has its respective basal volume. The illustrated configuration includes six devices 210, although more or less may be used, which may allow the pressure in the basal volume to be varied from one device 210 to the other.

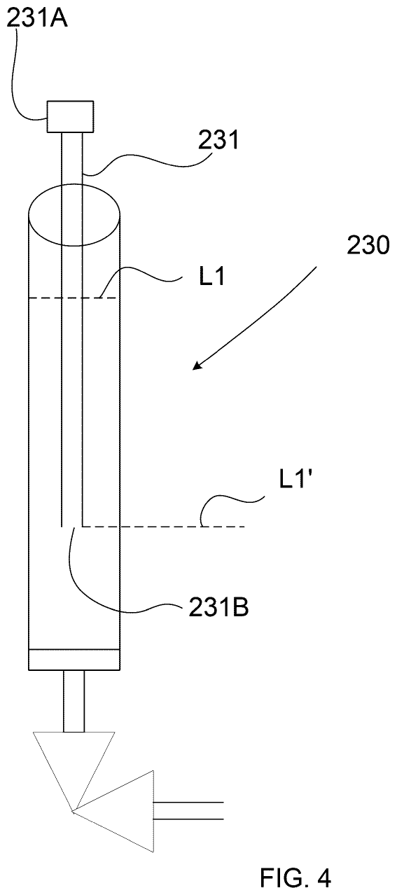

Referring now to FIG. 4, the source of the pressurized fluid may be a reservoir 230 containing a volume of the liquid. An air tube 231 may extend from a top end of the reservoir 230. The air tube 231 has a first end 231A exposed to atmospheric pressure and a second end 231B located below the first level L1 of liquid in the reservoir 230. A filter may cover the first end 231A of the air tube 231. The air tube 231 is used to move the first level L1 to an effective level L1′ that is aligned with the second end 231B of the air tube 231. Thus, regardless of the position of the first level L1 of the liquid in the reservoir 230, the effective height of liquid in the reservoir 230 corresponds to the effective level L1′ that is set by the air tube 231. This may provide a better control on the pressure generated on the basal side of the membrane. This configuration may prevent hydrostatic pressure inconsistency. In some embodiments, the space above the first level L1 may be sealed such that a pressure inside this space may be below atmospheric pressure. In a particular embodiment, therefore, a sealing mechanism is provided that may be adjusted to allow the sliding of the air tube, thereby permitting the effective pressure to be adjusted.

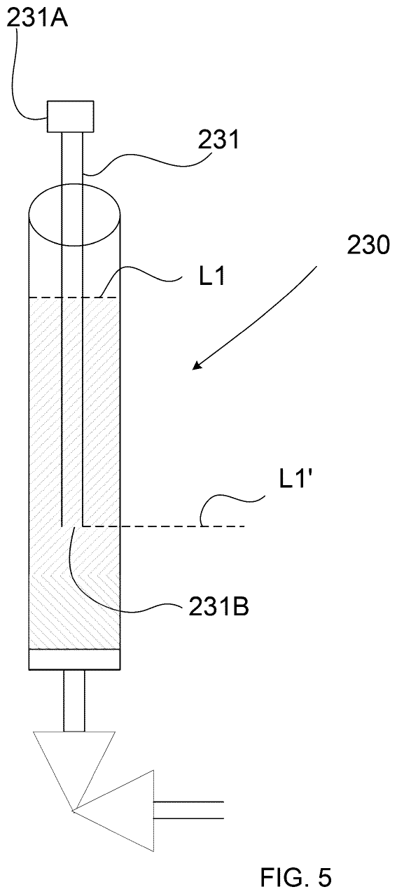

Referring to FIG. 5, in this embodiment, a second liquid different than the liquid supplied to the basal volume 25 may be used to generate hydrostatic pressure. The second liquid may be immiscible with the liquid of the basal volume 25 and has a density less than that of the liquid of the basal volume 25. This may permit savings on the liquid used for cell differentiation.

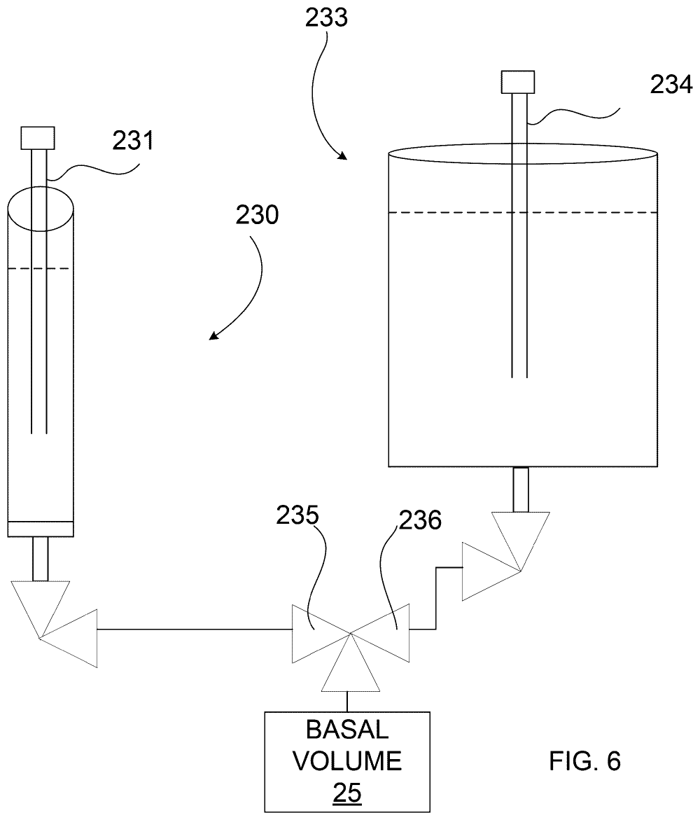

Referring to FIG. 6, in this embodiment, a second reservoir 233 containing the liquid is fluidly connectable to the basal volume 25 via suitable conduits and valves. The second reservoir 233 is equipped with a second air tube 234 to ensure a constant hydrostatic pressure. A volume of liquid contained in the second reservoir 233 is greater than that in the reservoir 230. The second reservoir 233 may be used as a source of liquid for replacing the liquid over time. The second air tube 234 may be used to maintain substantially constant the pressured applied on the basal side of the membrane during the change of the liquid. A first valve 235 may be used to selectively fluidly connect or disconnect the reservoir 230 from the basal volume 25. A second valve 236 may be used to selectively fluidly connect or disconnect the second reservoir 233 from the basal volume 25. The second reservoir 233 may keep the constant pressure throughout the entire culturing period and during a replacement of the liquid.

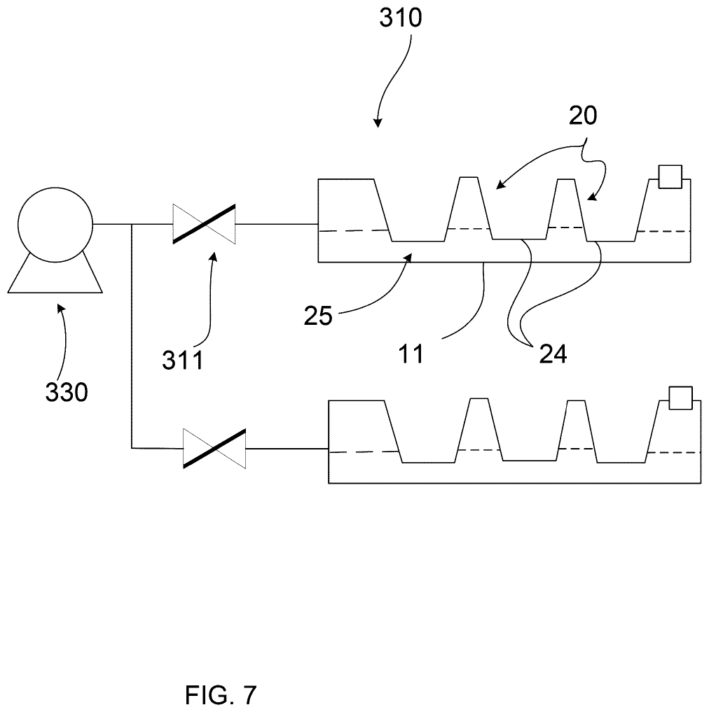

Referring now to FIG. 7, another device is shown at 310. For the sake of conciseness, only features differing from the device 210 of FIG. 2 are described below.

In the embodiment shown, the source of the pressurized fluid is a compressor 330 fluidly connectable to the basal volume 25 through a valve 311. The compressor 330 may be an air compressor able to provide pneumatic pressure in the basal volume 25 to increase a pressure on the basal side of the membranes 24. The compressor 330 may be replaced by a pump. In some embodiment, a pressurized gas tank may be used. This may reduce an amount of the liquid required for the culturing. The compressor 330 may be any suitable kind of compressor or pump, such as a piston pump, a diaphragm pump, a geared pump, a centrifugal pump, and so on.

As illustrated, it may be possible to connect a plurality of devices 310 in parallel to the compressor 330. Although two devices 310 are shown, the setup may include three or more devices.

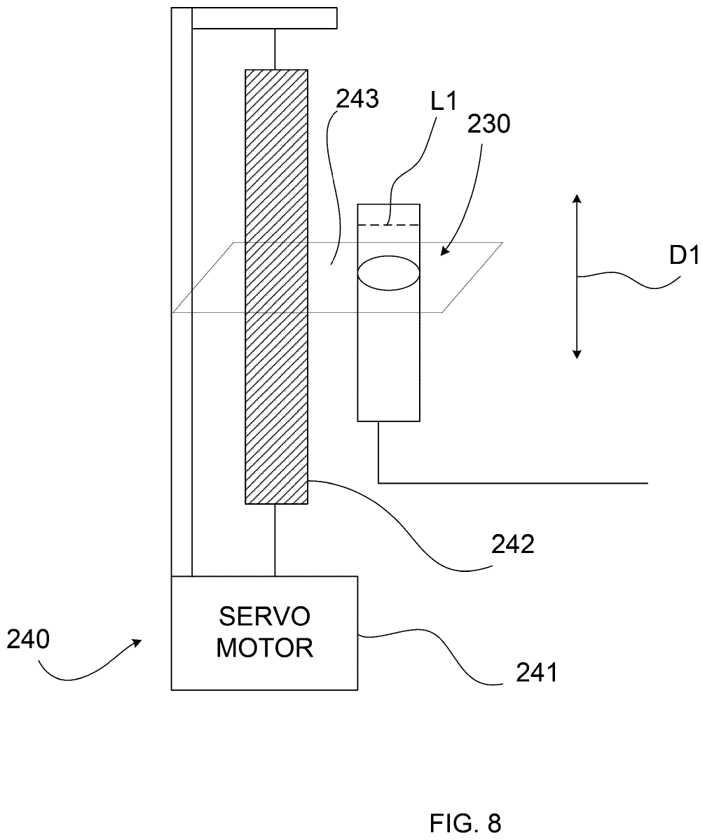

Referring to FIG. 8, in some cases, it may be desired to subject the membrane to a cyclically varying pressure. Hence, the reservoir 230 may be engaged to an actuator 240. The actuator 240 may include a motor 241 (e.g., electric motor) drivingly engaged to a threaded shank 242. The reservoir 230 may be secured to a member 243 threadingly engaged to the threaded shank 242. The member 243 may be prevented from rotating by any suitable means. The motor 241 may be powered to induce rotation of the threaded shank 242 in clockwise and counter clockwise direction to move the reservoir 230 up and down. More specifically, rotation of the threaded shank 242 includes a vertical movement of the reservoir 230 along direction D1, which is parallel to a rotation axis of the threaded shank 242, because of the threading engagement of the member 243 to the threaded shank 242. The member 243 may define an aperture being threaded to match threads of the threaded shank 242. This may effectively vary the height of the first level L1 of liquid in the reservoir 230 thereby increasing the hydrostatic pressure on the basal side of the membrane when the reservoir 230 is moved upward and decreasing the hydrostatic pressure on the basal side of the membrane when the reservoir 230 is moved downward.

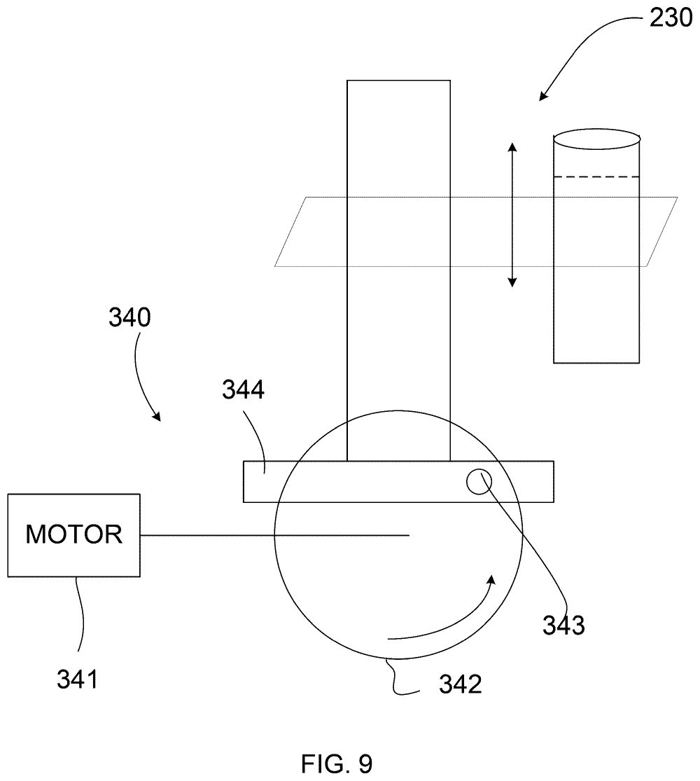

Referring now to FIG. 9, another actuator for varying the height of the reservoir 230 is shown at 340. The actuator 340 includes a motor 341 drivingly engaged to a rotating member 342. A pin 343 is disposed on the rotating member 342 and is offset from a rotation axis of the rotating member 342. A plate 344 is engaged to the pin 343. The plate 344 is secured to the reservoir 230. Therefore, powering the motor 341 induces rotation of the rotating member 342, and of the pin 343 about the rotation axis thereby moving the plate 344, and the reservoir 230 secured thereto, up and down. The plate 344 may define a slot within which the pin 343 rides to avoid the reservoir 230 from moving left to right in FIG. 9. Rotation of the rotating member 342 with the motor 341 induces a translation of the plate 344 and the reservoir 230 about an axis normal to the rotation axis of the rotating member 342.

The devices described above may be made of acrylic of any other suitable material. The cells that may be cultured with these devices include, for instance, lung cells, gut cells, pancreas cells, adipocytes cells, neuroendocrine cells, chondrocytes cells, and so on.

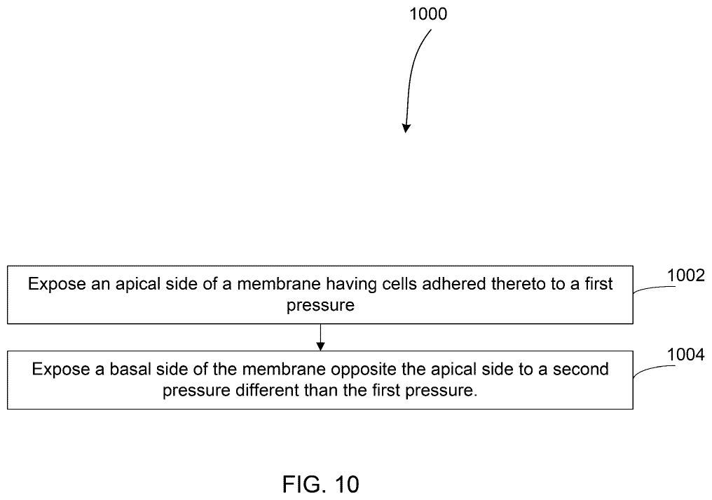

Referring now to FIG. 10, a method for cell differentiation is shown at 1000 and includes exposing the apical side of the membrane 24 having cells adhered thereto to a first pressure at 1002; and exposing the basal side of the membrane 24 opposite the apical side to a second pressure different than the first pressure. The second pressure may be greater than the first pressure. As discussed above, the second pressure may created by a tube or reservoir of the liquid having a level above a level of the liquid in the apical volume.

The method 1000 may include exposing both apical and basal sides of the membrane to the media at equal pressure, to allow a cell layer to form, before then the exposing of the apical and basal sides to a pressure differential. Thus, the apical and basal sides are exposed to the pressure differential once the cells layer become confluent.

In some embodiments, the exposing of the basal side of the membrane to the second fluid pressure includes fluidly connecting the basal volume to a source of a pressurized fluid.

The fluidly connecting of the basal volume to the source of the pressurized fluid may include fluidly connecting the basal volume to the reservoir of a liquid media. A first elevation of the liquid media in the reservoir is greater than a second elevation of the liquid media in the apical volume. Alternatively, the fluidly connecting of the basal volume to the source of the pressurized fluid includes fluidly connecting the basal volume to a compressor.

As explained above with reference to FIGS. 8-9, it may be possible to cyclically vary the second fluid pressure. This may be achieved by cyclically varying an elevation of the reservoir over time.

As shown in FIGS. 2-3, the exposing of the apical side of the membrane to the first fluid pressure and the exposing of the basal side of the membrane to the second fluid pressure includes exposing a plurality of apical sides of a plurality of membranes to the first fluid pressure and exposing a plurality of basal sides of the plurality of membranes to the second fluid pressure.

Results obtained by the inventors show significant improvements in phenotypes associated with differentiation for multiple cell lines including primary lung cells and miniature gut tissue. These initial results are very promising as it not only demonstrates that a pressure differential underlies ALI differentiation but also suggests an important potential role during fetal development in which air is also absent.

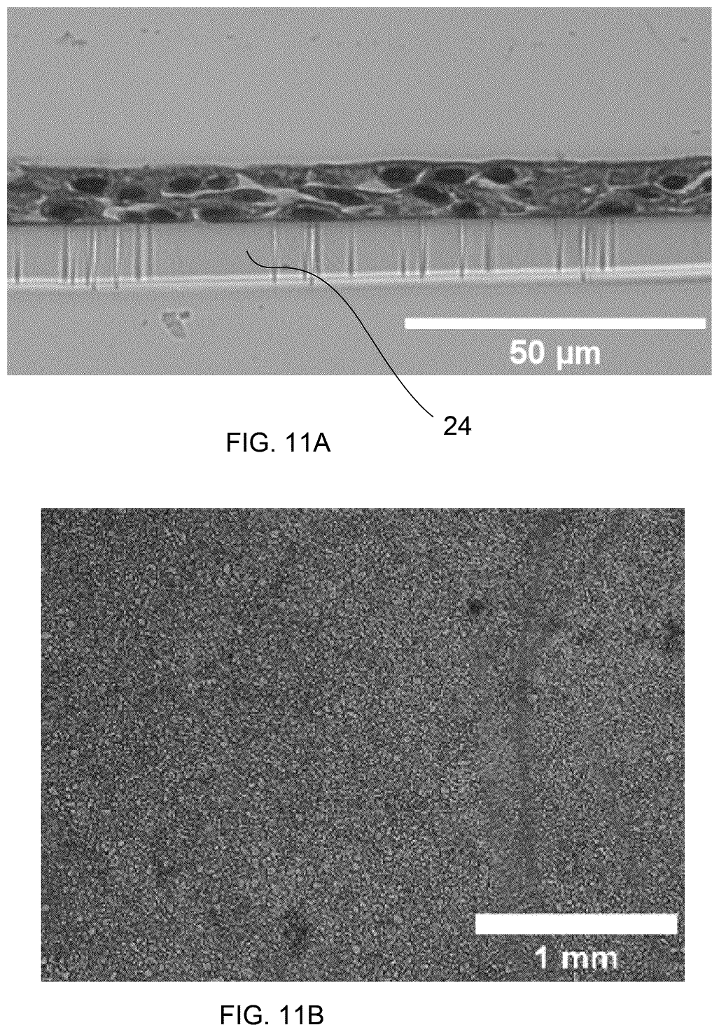

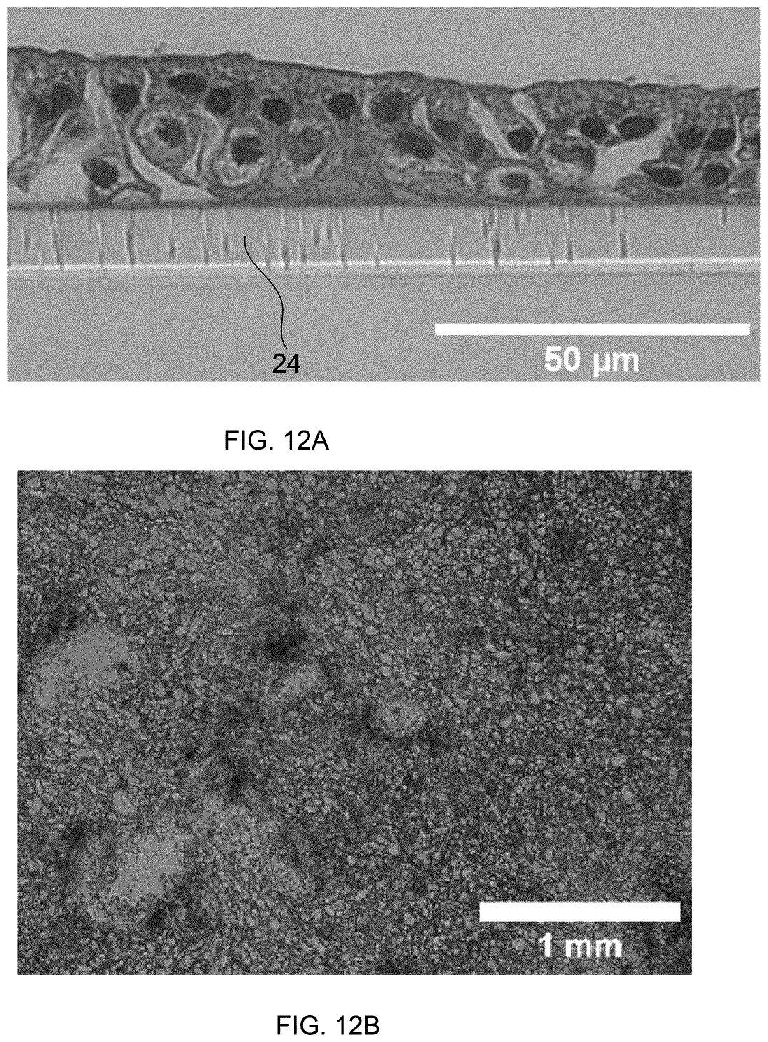

FIGS. 11A and 12A illustrate a primary lung cell line (human bronchial epithelial cells) while FIGS. 11B and 12B illustrate gut tissue (intestinal organoids). FIG. 12A illustrates that a thicker epithelial membrane is obtained with pressure difference on the membrane 24 compared to the membrane shown in FIG. 11A in which no pressure difference was applied. FIG. 12B shows that better heterogeneity is obtained with the pressure difference on the membrane compared to the membrane of FIG. 11B in which no pressure difference was applied. This suggest that differentiation towards different specialized cell types was obtain because of the pressure difference exerted on opposite sides of the membrane 24.

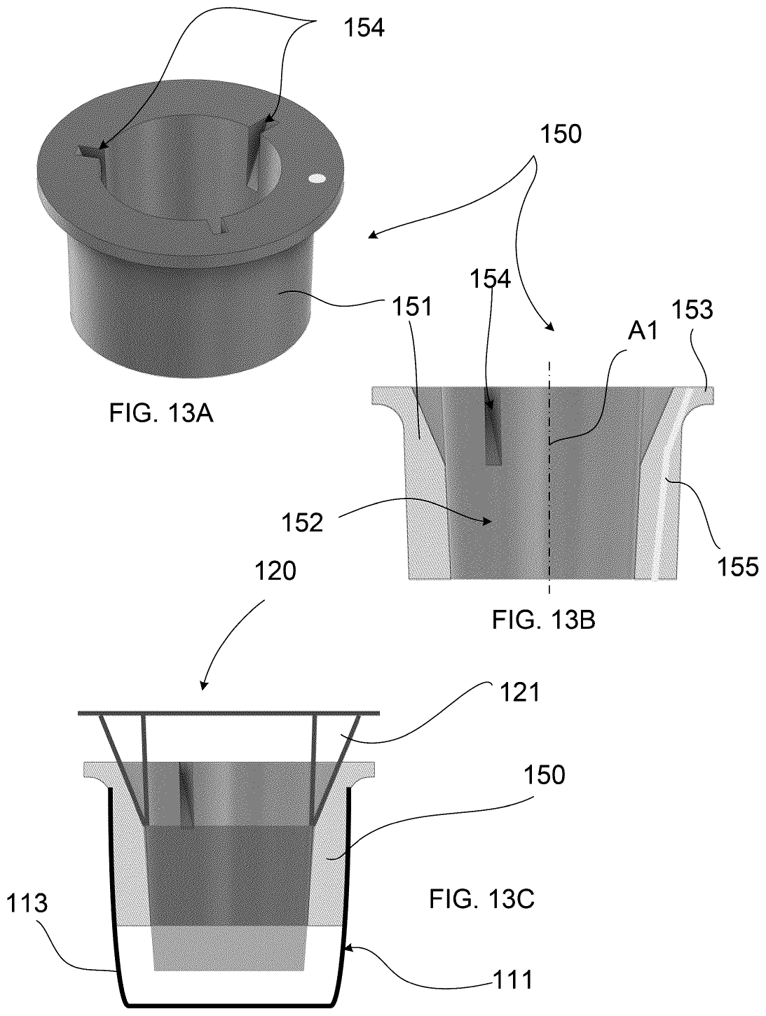

Referring to FIGS. 13A to 13C, an adaptor is shown at 150. The adaptor 150 is configured to be received within an enclosure 111 that contains the liquid media. The adaptor 150 is sealingly engaged to walls 113 of the enclosure 111. The adaptor 150 may be made of rubber or any other suitable material. The adaptor 150 creates an interface between the enclosure 111 and the insert 120. The adaptor 150 includes a peripheral wall 151 extending around a central axis A1 and surrounding a central passage 152. The central passage 152 extends from a top end of the adaptor 150 to a bottom end thereof. The adaptor 150 includes a flange 153, which may be omitted in some embodiments. The flange 153 may be used to abut against the walls 113 of the enclosure 111. The adaptor 150 defines notches 154, three in the embodiment shown, but more or less is contemplated. The notches 154 may be used to align the insert 120. The adaptor 150 defines a vent passage 155 extending within the peripheral wall 151 and extending from an inlet at the top end of the adaptor 150 to an outlet at the bottom end of the adaptor 150. The vent passage 155 may be sealed to prevent fluid communication between the volume located below the membrane of the insert 120 and an environment outside said volume.

As illustrated in FIG. 13C, the insert 120 is received within the central passage 152 of the adaptor 150 and ribs 121 of the insert 120 are slidably received within the notches 154 of the adaptor 150. An outer face of the peripheral wall 151 is in abutment against an inner face of the walls 113 of the enclosure 111 to create a sealing engagement between the adaptor 150 and the enclosure 111. The adaptor 150 is thus sized to mate with the enclosure 111 while being shaped to accommodate the insert 120.

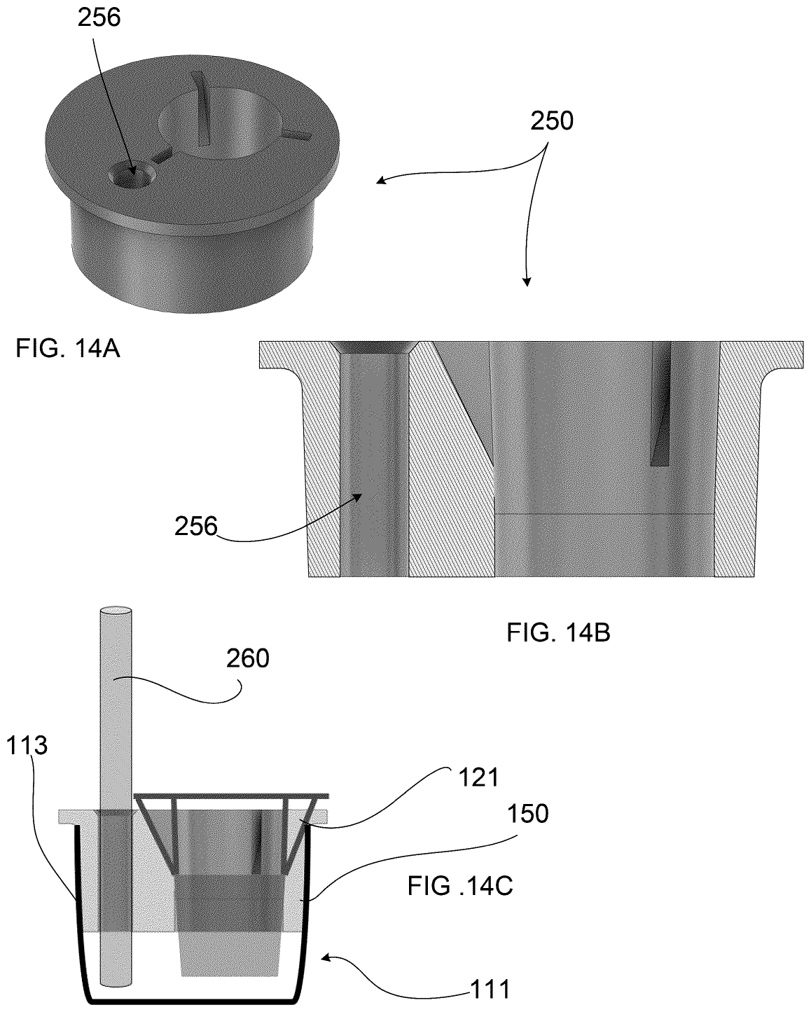

Referring now to FIGS. 14A-14C, another embodiment of an adaptor is shown at 250. For the sake of conciseness, only features differing from the adaptor 150 of FIGS. 13A-13C are described below.

In the embodiment shown, the adaptor 250 defines a tube-receiving passage 256 sized to receive a tube (e.g., capillary tube) 260. A sealing engagement may be provided between an outer surface of the tube 260 and an inner face bounding the tube-receiving passage 256. The tube 260 may be filled with the liquid media to increase a pressure acting against the membrane of the insert 120.

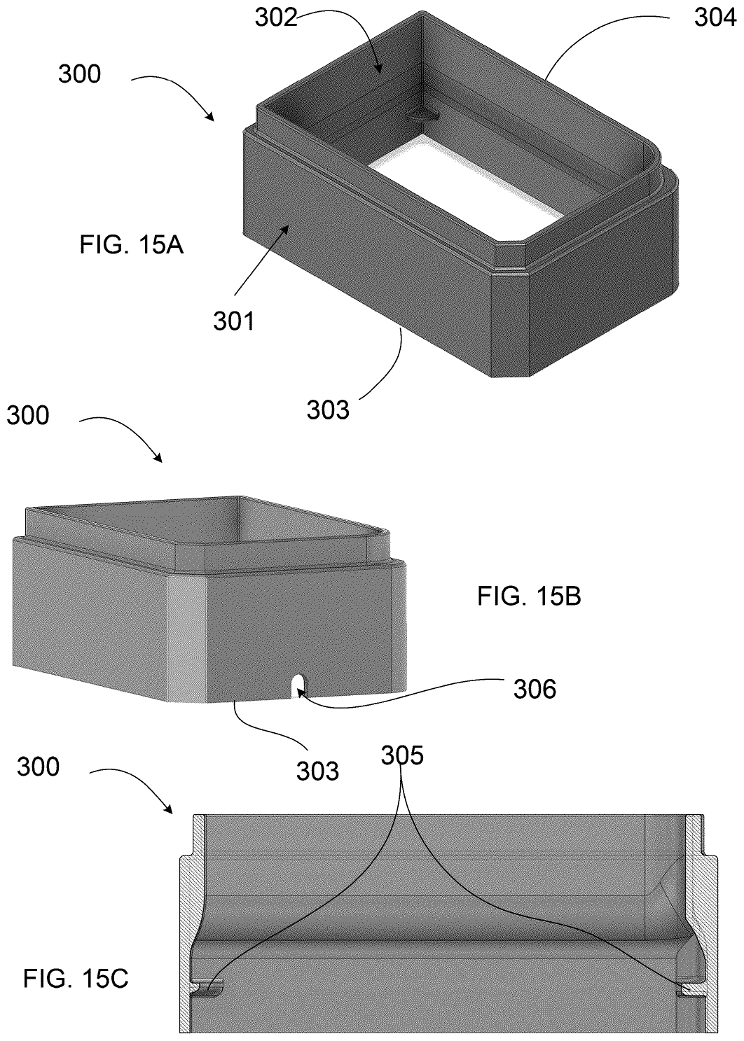

Referring to FIGS. 15A to 15C, a lid extender is shown at 300. The lid extender 300 is configured to receive devices 210 as described above with reference to FIG. 3. It is used when it is required to maintain sterility of the content of the devices 210. The lid extender 300 has a peripheral wall 301 extending around a volume 302 sized for receiving the devices 210. The peripheral wall 301 extends from a bottom end 303 to a top end 304. The peripheral wall 301 has a substantially rectangular shape, but any suitable shapes (e.g., square, round, etc) is contemplated.

As shown in FIG. 15C, the lid extender 300 has protrusions 305, four in this embodiment but more or less may be used, extending inwardly from the peripheral wall 301 into the volume 302. The protrusions 305 are configured to hold the lid extender 300 against top plate of the devices 210.

As shown in FIG. 15B, the lid extender 300 has an opening 306 extending through the peripheral wall 301 at the bottom end 303 thereof. The opening 306 may be U-shaped, but other shapes are contemplated. The opening 306 is configured for providing access to the tubes used to increase the pression in the enclosures.

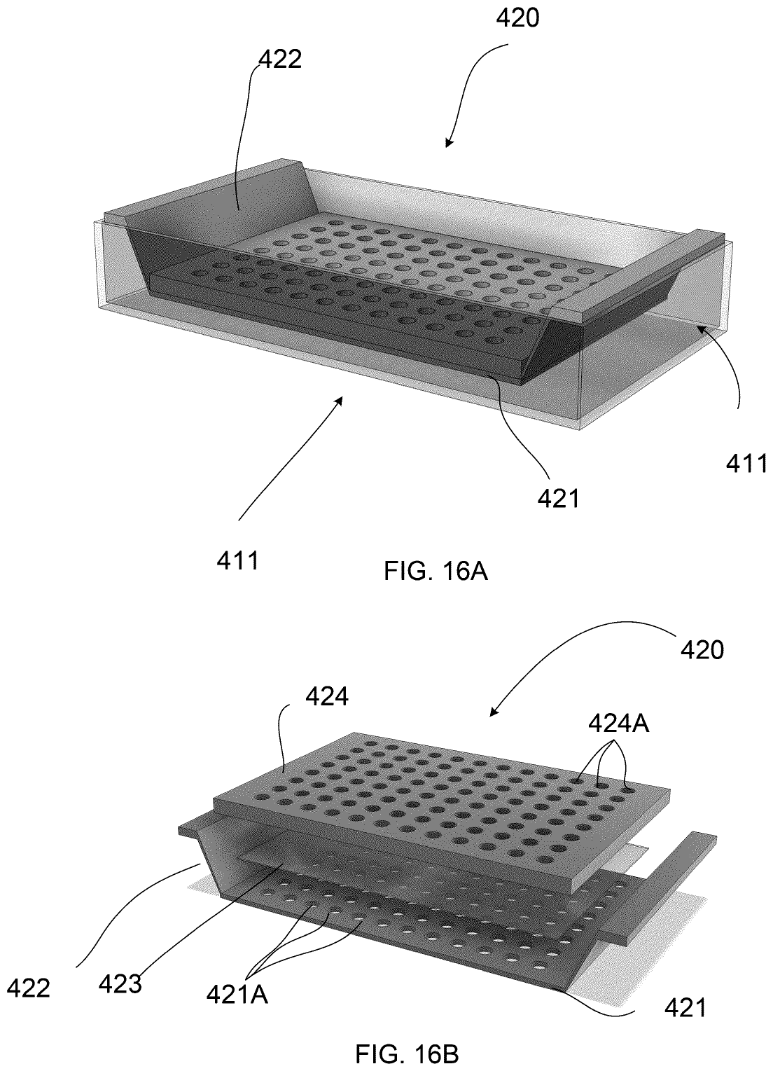

Referring to FIGS. 16A-16B, an insert in accordance with another embodiment is shown at 420. The insert 420 may be used in conjunction with the lid extender 300 described above. The insert 420 is received within an enclosure 411. The insert 420 includes a plate 421 and side walls 422 protruding upwardly from the plate 421. The plate 421 defines a plurality of apertures 421A. A porous membrane 423 is disposed over the plate 421. A chamber module 424 is disposed over the porous membrane 423 such that the porous membrane 423 is disposed between the chamber module 424 and the plate 421. The chamber module 424 defines a plurality of chambers 424A each being in register with a respective one of the apertures 421A defined through the plate 421.

In this embodiment, the chamber module 424 may be tightly pressed against the porous membrane 423, which is thus itself pressed against the plate 421 thereby creating cell culture chambers with a bottom membrane. In this embodiment, a matrix of 8×12 chambers is provided, but more or less is contemplated.

The insert 420 and enclosure 411 may be operatively connected to a pressure generating device as illustrated in FIG. 7. The pressure generating device may be a pump, or, alternatively, may be a tube containing the liquid media at an elevation selected to ensure the desired pressure differential across the membrane 423.

The disclosed device provides a differentiation method and system which may be used to improve upon, or replace, to replace currently-employed air-liquid interface systems and methods. The present differentiation method and system may provide a more robust, flexible and scalable process.

The disclosed device may provide a custom cell culture well plate compatible with existing ALI culturing apparatus, may characterize differentiation timelines and outcomes for multiple cell lines, and may be used to perform RNA sequencing analysis on various cell lines to identify the crucial genes involved in differentiation, and identify a chemical-based differentiation method based on this analysis.

In the context of the present disclosure, expressions “above”, “below”, “top”, and “bottom” are in reference to an elevation relative to a ground surface.

The embodiments described in this document provide non-limiting examples of possible implementations of the present technology. Upon review of the present disclosure, a person of ordinary skill in the art will recognize that changes may be made to the embodiments described herein without departing from the scope of the present technology. Yet further modifications could be implemented by a person of ordinary skill in the art in view of the present disclosure, which modifications would be within the scope of the present technology.

1. A device for cell differentiation, comprising:
an enclosure having a top and a bottom, the enclosure defining a liquid-receiving volume for receiving a liquid media;
an insert received in the enclosure, the insert having:
a peripheral wall circumscribing an apical volume and extending from a top end proximate the top of the enclosure to a bottom end, an opening defined at the bottom end of the insert, and
a membrane extending across the opening and defining pores sized to receive cells, the membrane having a basal side facing the bottom of the enclosure and an apical side opposite the basal side, the apical side adapted to receive cells thereon, the membrane and the peripheral wall separating the liquid-receiving volume into an apical volume within the insert and a basal volume below the membrane within the enclosure; and
a source of a pressurized fluid in fluid flow communication with the basal volume within the enclose, the pressurized fluid being at a pressure selected such that a basal pressure in the basal volume acting against the basal side of the membrane is greater than an apical pressure in the apical volume acting against the apical side of the membrane.

2. The device of claim 1, wherein the source of the pressurized fluid is a reservoir of the liquid media, the reservoir in fluid flow communication with the basal volume, a first elevation of the liquid media in the reservoir being greater than a second elevation of the liquid media in the apical volume.

3. The device of claim 2, wherein the reservoir is engaged to an actuator operable to vary the elevation of the reservoir.

4. The device of claim 3, wherein the actuator includes a motor drivingly engaged to a threaded shank and a member threadingly engaged to the threaded shank, the reservoir mounted to the member, rotation of the threaded shank with the motor inducing a translation of the member and the reservoir about a rotation axis of the threaded shank.

5. The device of claim 3, wherein the actuator includes a motor drivingly engaged to a rotating member for rotation about a rotation axis, the reservoir mounted to the rotating member, the rotating member having a pin secured thereto and offset from the rotation axis of the rotating member, the pin slidably received within a slot defined by the member, rotation of the rotating member with the motor induces a translation of the member and the reservoir about an axis normal to the rotation axis of the rotating member.

6. The device of claim 1, wherein the source of the pressurized fluid is a compressor fluidly connected to the basal volume and configured for increasing an air pressure in the basal volume.

7. The device of claim 1, wherein the enclosure defines an outlet in fluid flow communication with the basal volume.

8. The device of claim 7, comprising a valve in fluid flow communication with the outlet, the valve configured for selectively fluidly connected the basal volume to an environment outside the basal volume.

9. The device of claim 1, wherein the insert includes a plurality of inserts disposed within the basal volume of the enclosure, each of the plurality of inserts defining a respective apical volume.

10. The device of claim 9, wherein two or more of the plurality of inserts have different basal volumes, such as to induce different pressures pressure on a basolateral side of the membrane.

11. The device of claim 9, comprising a top plate secured to the top of the enclosure, the top plate defines apertures, the plurality of inserts received through the apertures.

12. A method for culturing cells, comprising:
exposing an apical side of a membrane having cells adhered thereto to a first fluid pressure; and
exposing a basal side of the membrane opposite the apical side to a second fluid pressure different than the first fluid pressure.

13. The method of claim 12, wherein the second fluid pressure is greater than the first fluid pressure.

14. The method of claim 13, further comprising, prior to the exposing the basal side of the membrane to the second fluid pressure, exposing both the apical side and the basal side to media at substantially equal pressure, determining when a cell layer on the membrane becomes confluent, and then exposing the basal side of the membrane to the second fluid pressure.

15. The method of claim 12, wherein the membrane extends across an opening defined by an insert received within a fluid-receiving volume of an enclosure, the membrane dividing the fluid-receiving volume in an apical volume and a basal volume below the apical volume, the exposing of the basal side of the membrane to the second fluid pressure includes fluidly connecting the basal volume to a source of a pressurized fluid.

16. The method of claim 15, wherein the fluidly connecting of the basal volume to the source of the pressurized fluid includes fluidly connecting the basal volume to a reservoir of a liquid media, a first elevation of the liquid media in the reservoir being greater than a second elevation of the liquid media in the apical volume.

17. The method of claim 15, wherein the fluidly connecting of the basal volume to the source of the pressurized fluid includes fluidly connecting the basal volume to a compressor.

18. The method of claim 16, comprising cyclically varying the second fluid pressure.

19. The method of claim 18, wherein the cyclically varying the second fluid pressure includes cyclically varying an elevation of the reservoir.

20. The method of claim 12, wherein the exposing of the apical side of the membrane to the first fluid pressure and the exposing of the basal side of the membrane to the second fluid pressure includes exposing a plurality of apical sides of a plurality of membranes to the first fluid pressure and exposing a plurality of basal sides of the plurality of membranes to the second fluid pressure.
